SEQUENCE LISTING (8 sequences)

SEQ ID NO: 1 - protein, 30 aa, synthetic construct
HAEGTFTSDVSSYLEGQAAKEFIAWLVKGR

SEQ ID NO: 2 - protein, 31 aa, synthetic construct
HGEGTFTSDVSSYLEGQAAKEFIAWLVKGRG

SEQ ID NO: 3 - protein, 66 aa, synthetic construct
HAEGTFTSDVSSYLEGQAAKEFIAWLVKGRGGGGSGGGGSGGGGSRRPKGRGKRRRENQRPTDCHL

SEQ ID NO: 4 - protein, 66 aa, synthetic construct
RRPKGRGKRRRENQRPTDCHLGGGGSGGGGSGGGGSHAEGTFTSDVSSYLEGQAAKEFIAWLVKGR

SEQ ID NO: 5 - DNA, 93 bp, synthetic construct
  source 1..93: mol_type = other DNA
cacgccgagggcacatttacaagcgacgtgtccagctacctggaaggccaggccgctaaagagttcatcgcctggctggtgcggggaaggggc

SEQ ID NO: 6 - DNA, 93 bp, synthetic construct
  source 1..93: mol_type = other DNA
catggtgaagggacctttaccagtgatgtaagttcttatttggaaggccaagctgccaaggaattcattgcttggctggtgaaaggccgagga

SEQ ID NO: 7 - DNA, 198 bp, synthetic construct
  source 1..198: mol_type = other DNA
catgctgaagggacctttaccagtgatgtaagttcttatttggaaggccaagctgccaaggaattcattgcttggctggtgaaaggccgaggtggaggcggttcaggcggaggtggctctggcggtggcggatcgaggagacccaagggcagggggaagaggaggagagagaaccagagacccacagactgccacctg

SEQ ID NO: 8 - DNA, 198 bp, synthetic construct
  source 1..198: mol_type = other DNA
aggagacccaagggcagggggaagaggaggagagagaaccagagacccacagactgccacctgggtggaggcggttcaggcggaggtggctctggcggtggcggatcgcatgctgaagggacctttaccagtgatgtaagttcttatttggaaggccaagctgccaaggaattcattgcttggctggtgaaaggccga

(19) United States
(12) Patent Application Publication
(10) Pub. No.: US 2026/0049337 A1
(43) Pub. Date: Feb. 19, 2026

(54) A REPLICATIVE ONCOLYTIC ADENOVIROUS CAPABLE OF INHIBITING TUMOR PROGRESSION AND PROLONGING SURVIVAL TIME IN TUMOR-BEARING INDIVIDUALS AND USE THEREOF

(71) Applicant: NANJING VIROTHER BIOPHARMACEUTICAL CO., LTD., Nanjing, Jiangsu (CN)

(72) Inventor: Jiwu WEI, Nanjing (CN)

(21) Appl. No.: 19/102,321

(22) PCT Filed: Aug. 8, 2022
(86) PCT No.: PCT/CN2022/110988

§ 371 Date: Feb. 7, 2025

(51) Int. Cl. C12N 15/861; A61K 9/00; A61K 35/761; A61K 48/00; A61P 35/04; C07K 14/605; C12N 7/00

(52) U.S. Cl. CPC C12N 15/861; A61K 9/0019; A61K 35/761; A61K 48/005; A61P 35/04; C07K 14/605; C12N 7/00; C07K 2319/02; C07K 2319/33; C12N 2710/10043

(57) ABSTRACT

Provided are a recombinant adenovirus or adenoviral vector expressing GLP1 receptor agonist that shows expected anti-tumor effects and prolonged survival time in tumor-bearing subjects and the pharmaceutical composition comprising the same.

### SEQUENCE LISTING

This application contains an ST.26 compliant Sequence Listing, which is submitted in xml format via Patent Center and is hereby incorporated by reference in its entirety. The .xml copy, created on Nov. 22, 2023, is named 147666-8002WO00 Sequence Listing.xml and is 8,621 bytes in size.

### TECHNICAL FIELD

The present disclosure relates to the field of tumor therapy using recombinant adenovirus or adenoviral vectors.

### BACKGROUND

Cancer has become the number one disease that endangers human life and health, with about 4 million new cancer patients and nearly 3 million deaths from cancer in China each year. Surgery is the most effective treatment for early-stage cancer, but it does not significantly improve the great majority of patients with advanced and metastatic cancer. Although radiotherapy/chemotherapy and other adjuvant therapies can suppress tumor progression to some extent, the overall survival rate of cancer patients has not improved significantly. The search for new, safe and effective therapeutic agents is a hot topic in pharmacological research worldwide today.

Tumor immunotherapy has gained much attention in recent years because of its remarkable efficacy. Tumor immunotherapy is a treatment that activates the body's immune system to kill and monitor cancer cells through activation and/or modulation of immune-related pathways. Oncolytic virus is a rising star in the field of tumor immunotherapy, and oncolytic virus-mediated antitumor immunotherapy has received increasing attention with the marketing approval of oncolytic virus T-Vec by FDA in late 2015. Oncolytic virus is a class of viruses with the ability to self-replicate and replicate in large numbers within tumor cells, and have a variety of unique antitumor effects: they can induce immunogenic death of tumor cells; activate the body's immune surveillance; and express recombinant proteins to modulate the tumor microenvironment. Several clinical trials related to oncolytic virus are currently underway, such as adenovirus, herpes simplex, measles virus, poxvirus, vesicular stomatitis virus, etc., and good therapeutic results have been achieved.

However, solid tumors escape the body's immune surveillance and promote invasion and metastasis through multiple mechanisms including abnormal metabolism, pro-inflammatory microenvironment and the like. Studies have found that the oncolytic virus would upregulate the tumor-promoting inflammation in the tumor microenvironment and alter the metabolic microenvironment while activating immunity, thereby adversely affecting their anti-tumor effects. Disclosed herein are novel oncolytic virus constructs and their use in antitumor therapy.

### SUMMARY

One aspect of the disclosure relates to a recombinant adenovirus or adenoviral vector comprising a first promoter, an E1A early activation replication element, an optional second promoter, and a target nucleotide (e.g., DNA) sequence encoding a target protein comprising a glucagon-like peptide-1 (GLP-1) receptor agonist, the first promoter being a constitutive promoter; and the optional second promoter being a constitutive promoter which may be the same as or different from the first promoter, with the proviso that when the target protein is GLP-1(7-36), the Ad5 adenovirus or adenoviral vector comprises two promoters.

Another aspect of the disclosure relates to pharmaceutical composition comprising the recombinant adenovirus or adenoviral vector disclosed herein, and a pharmaceutically acceptable excipient.

Another aspect of the disclosure relates to uses of the recombinant adenovirus or adenoviral vector disclosed herein or pharmaceutical composition comprising same for antitumor treatment and/or prevention in a subject. In certain embodiments, the antitumor treatment and/or prevention comprising inducing CD8+T cell-mediated antitumor effects in the subject.

Another aspect of the disclosure relates to preparation of the recombinant adenovirus or adenoviral vector disclosed herein.

### BRIEF DESCRIPTION OF THE DRAWINGS

FIG. 1A shows an embodiment of a single-promoter shuttle vector for preparation of an embodiment of the recombinant adenovirus or adenoviral vector expressing GLP-1 disclosed herein.

FIG. 1B shows an embodiment of a DNA construct comprising in a single-promoter shuttle vector for preparation of an embodiment of the recombinant adenovirus or adenoviral vector expressing GLP-1 analogue (e.g., AdV-GLP-1(7-37/A8G)-CMV) according to Example I, IL-2 SS: a DNA sequence encoding a IL-2 signal peptide: MDWTWRILFLVAAATGAHS, and GLP1 analogue being a DNA sequence encoding GLP-1(7-37/A8G), e.g., as shown in Table 1.

FIG. 1C shows another embodiment of a single-promoter shuttle vector for preparation of an embodiment of the recombinant adenovirus or adenoviral vector expressing GLP-1 disclosed herein.

FIG. 2A shows the construction of an embodiment of a two-promoter shuttle plasmid.

FIG. 2B shows an example of the DNA sequence comprising CMV and EF1a for a double-promoter shuttle vector for AdV-GLP-1(7-36)-CMV/EF1a, and an example of DNA sequence encoding GLP-1(7-36).

FIG. 2C shows an example of the DNA sequence comprising CMV and EF1a for a double-promoter shuttle vector for AdV-GLP-1(7-36)-PLGF2-CMV/EF1a.

FIG. 2D shows an example of the DNA sequence comprising CMV and EF1a for a double-promoter shuttle vector for AdV-PLGF2-GLP1(7-36)-CMV/EF1a.

FIG. 2E shows an example of the construction of another embodiment of a two-promoter shuttle plasmid.

FIG. 3A shows an ELISA evaluation of expression and secretion function of one embodiment of recombinant adenovirus expressing GLP1 (7-36) as shown in Example II.

FIG. 3B shows the viral replication capacity in pancreatic carcinoma cells of one embodiment of the recombinant adenovirus expressing GLP1 (Ad5_G01) was higher than control adenovirus that did not express GLP1 (Ad5_con).

FIG. 3C shows the viral replication capacity in colorectal carcinoma cells of one embodiment of recombinant adenovirus expressing GLP1 (Ad5_G01) was higher than control adenovirus that does not express GLP1 (Ad5_con).

FIG. 4 shows an embodiment of recombinant adenovirus expressing a GLP1 analogue (ADV GLP1 (7-37/A8G)) generated biological active form of GLP1, as shown in Example II.

FIG. 5 shows an embodiment of recombinant adenovirus expressing GLP1 (7-36) generated biological active form of GLP1, as shown in Example II.

FIG. 6 shows the experiment design of evaluation of an embodiment of a recombinant adenovirus expressing GLP1(7-36) in pancreatic carcinoma mouse model, e.g., Example III(A).

FIG. 7A shows the tumor growth data of negative control group treated with PBS in pancreatic carcinoma mouse model as shown in FIG. 6 (e.g., Example III(A)).

FIG. 7B shows the tumor growth data of the AdV-Ctrl group treated with control adenovirus that does not express a target peptide in pancreatic carcinoma mouse model as shown in FIG. 6 (e.g., Example III(A)).

FIG. 7C shows the tumor growth data of the AdV-GLP1 (7-36) group treated with an embodiment of a recombinant adenovirus expressing GLP1(7-36) in pancreatic carcinoma mouse model as shown in FIG. 6 (e.g., Example III(A)).

FIG. 8 shows that in pancreatic carcinoma mouse model as shown in FIG. 6 (e.g., Example III(A)), the survival rate in the group treated with an embodiment of a recombinant adenovirus expressing GLP1(7-36) (AdV-GLP1(7-36)) significantly increased compared to the group treated with control adenovirus that does not express a target peptide (AdV-Ctrl) and the negative group treated with PBS (PBS), respectively, p<0.05.

FIG. 9 shows that in pancreatic carcinoma mouse model as shown in FIG. 6 (e.g., Example III(A)), no significant difference was shown in the body weights among the groups treated with an embodiment of a recombinant adenovirus expressing GLP1(7-36) (AdV-GLP1(7-36)), control adenovirus that does not express a target peptide (AdV-Ctrl), and the negative group treated with PBS (PBS), respectively.

FIG. 10 shows the experiment design of evaluation of an embodiment of a recombinant adenovirus expressing GLP1(7-36) in an orthotopic pancreatic carcinoma mouse model, e.g., Example III(A).

FIG. 11A shows the total luciferase counts measured among groups of orthotopic Panc02-car mouse model as shown in FIG. 10 treated with an embodiment of a recombinant adenovirus expressing GLP1(7-36) (AdV-GLP1(7-36)), control adenovirus that does not express a target peptide (AdV-Ctrl), or the negative group treated with PBS (PBS), e.g., Example III(A).

FIG. 11B shows the tumor volume measured by luminescence among groups of orthotopic Panc02-car mouse model as shown in FIG. 10 treated with an embodiment of a recombinant adenovirus expressing GLP1(7-36) (AdV-GLP1(7-36)), control adenovirus that did not express a target peptide (AdV-Ctrl), or the negative group treated with PBS (PBS), e.g., Example III(A).

FIG. 12 shows that in orthotopic Panc02-car mouse model as shown in FIG. 10 (e.g., Example III(A)), the survival rate in the group treated with an embodiment of a recombinant adenovirus expressing GLP1(7-36) (AdV-GLP1(7-36)) significantly increased compared to the group treated with control adenovirus that does not express a target peptide (AdV-Ctrl) and the negative group treated with PBS (PBS), respectively, p<0.05.

FIG. 13 shows the experiment design of evaluation of an embodiment of a recombinant adenovirus expressing GLP1(7-36) in colorectal carcinoma mouse model, e.g., Example III(B).

FIG. 14A shows the tumor growth data of negative control group treated with PBS in colorectal carcinoma mouse model as shown in FIG. 13 (e.g., Example III(B)).

FIG. 14B shows the tumor growth data of the AdV-Ctrl group treated with control adenovirus that does not express a target peptide in colorectal carcinoma mouse model as shown in FIG. 13 (e.g., Example III(B)).

FIG. 14C shows the tumor growth data of the AdV-GLP1 (7-36) group treated with an embodiment of a recombinant adenovirus expressing GLP1(7-36) in colorectal carcinoma mouse model as shown in FIG. 13 (e.g., Example III(B)).

FIG. 15 shows that in a colorectal carcinoma mouse model as shown in FIG. 13 (e.g., Example III(B)), the survival rate in the group treated with an embodiment of a recombinant adenovirus expressing GLP1(7-36) (AdV-GLP1(7-36)) significantly increased compared to the group treated with control adenovirus that does not express a target peptide (AdV-Ctrl) (p<0.01) and the negative group treated with PBS (PBS) (p<0.05), respectively.

FIG. 16 shows that in a colorectal carcinoma mouse model as shown in FIG. 13 (e.g., Example III(B)), no significant difference was shown in the body weights among the groups treated with an embodiment of a recombinant adenovirus expressing GLP1(7-36) (AdV-GLP1(7-36)), control adenovirus that does not express a target peptide (AdV-Ctrl), and the negative group treated with PBS (PBS), respectively.

FIG. 17 shows the experiment design of evaluation of an embodiment of a recombinant adenovirus expressing GLP1(7-36) in a melanoma mouse model, e.g., Example III(C).

FIG. 18 shows that in a melanoma mouse model as shown in FIG. 17 (e.g., Example III(C)), the survival rate in the group treated with an embodiment of a recombinant adenovirus expressing GLP1(7-36) (AdV-GLP1(7-36)) significantly increased compared to the group treated with control adenovirus that does not express a target peptide (AdV-Ctrl) (P=0.0014) and the negative group treated with PBS (PBS) (P=0.0015), respectively.

FIG. 19 shows the experiment design of evaluation of an embodiment of a recombinant adenovirus expressing GLP1(7-36) in a glioma mouse model, e.g., Example III(D).

FIG. 20 shows that in a glioma mouse model as shown in FIG. 19, the tumor growth rate in the group treated with an embodiment of a recombinant expressing GLP1(7-36) (AdV-GLP-1(7-36)) was statistically significantly slower compared to the group treated with control adenovirus that does not express a target peptide (AdV-Ctrl) (p=0.0001) and the negative group treated with PBS (PBS) (P=0.0001), respectively.

FIG. 21 shows that in a glioma mouse model as shown in FIG. 19 (e.g., Example III(D)), the survival rate in the group treated with an embodiment of a recombinant adenovirus expressing GLP1(7-36) (AdV-GLP1(7-36)) significantly increased compared to the group treated with control adenovirus that does not express a target peptide (AdV-Ctrl) (p=0.0027) and the negative group treated with PBS (PBS) (p=0.0082), respectively.

FIG. 22 shows the experiment design of evaluation of an embodiment of a recombinant adenovirus expressing GLP1(7-36) in a kidney cancer mouse model, e.g., Example III(E).

FIG. 23A shows the tumor growth data of negative control group treated with PBS in the kidney cancer mouse model as shown in FIG. 22 (e.g., Example III(E)).

FIG. 23B shows the tumor growth data of the AdV-Ctrl group treated with control adenovirus that does not express a target peptide in the kidney cancer mouse model as shown in FIG. 22 (e.g., Example III(C)).

FIG. 23C shows the tumor growth data of the AdV-GLP1 (7-36) group treated with an embodiment of a recombinant adenovirus expressing GLP1(7-36) in the kidney cancer mouse model as shown in FIG. 22 (e.g., Example III(C)).

FIG. 23D shows that in a kidney cancer mouse model as shown in FIG. 22, the tumor growth rate in the group treated with an embodiment of a recombinant expressing GLP1(7-36) (AdV-GLP-1(7-36)) was statistically significantly slower compared to the group treated with control adenovirus that does not express a target peptide (AdV-Ctrl) (p<0.0001) and the negative group treated with PBS (PBS) (p<0.05), respectively.

FIG. 24 shows that in the pancreatic cancer mouse model (e.g., Example IV(A)), the group treated with an embodiment of a recombinant expressing GLP1(7-36) (AdV-GLP-1(7-36)) and the groups treated with anti-CD4 (GLP1(7-36)+anti-CD4) or anti-NK (GLP1(7-36)+anti-NK) before the AdV-GLP-1(7-36)-CMV/EF1a treatment showed slower tumor growth rate than the group treated with anti-CD8 (GLP1(7-36)+anti-CD8) before the AdV-GLP-1(7-36) treatment, the control Ad5 (AdV-Ctrl) and the negative control group treated with PBS only (PBS).

FIG. 25 shows that in the pancreatic cancer mouse model (e.g., Example IV(A)), the group treated with anti-CD8 (GLP1(7-36)+anti-CD8 before the AdV-GLP-1(7-36)-CMV/EF1a treatment did not show statistical significance in survival rate compared with the group treated with the control Ad5 (AdV-Ctrl) but showed statistically significantly lower survival rate compared to the group treated with AdV-GLP-1(7-36)-CMV/EF1a only (AdV-GLP1(7-36)). The group treated with AdV-GLP-1(7-36)-CMV/EF1a only (AdV-GLP1(7-36)) and the groups treated with anti-CD4 (GLP1(7-36)+anti-CD4) or anti-NK (GLP1(7-36)+anti-NK) before the AdV-GLP-1(7-36)-CMV/EF1a treatment showed statistically significantly longer survival rate compared to the group treated with the control Ad5 (*: p<0.5, AdV-Ctrl). The groups treated with anti-CD4 (GLP1(7-36)+anti-CD4) or anti-NK (GLP1(7-36)+anti-NK) before the AdV-GLP-1(7-36)-CMV/EF1a treatment did not show statistically significant difference in survival rate compared to the group treated with AdV-GLP-1(7-36)-CMV/EF1a only (AdV-GLP1(7-36)).

FIG. 26A shows the percentages of IFN-γ+ CD8+ T cells among Adv-GLP1 (7-36) group, AdV-Ctrl group, and PBS group in pancreatic carcinoma mouse model as shown in Example IV(B).

FIG. 26B shows the percentages of GZMB+ CD8+ T cells among Adv-GLP1 (7-36) group, AdV-Ctrl group, and PBS group in pancreatic carcinoma mouse model as shown in Example IV(B).

FIG. 26C shows the percentages of PD1+ CD8+ T cells among Adv-GLP1 (7-36) group, AdV-Ctrl group, and PBS group in pancreatic carcinoma mouse model as shown in Example IV(B).

FIG. 27A shows LCN2 expression in CD8+ T cells among Adv-GLP1 (7-36) group, AdV-Ctrl group, and PBS group in pancreatic carcinoma mouse model as shown in Example IV(B).

FIG. 27B shows LCN2 expression in CD4 T cells among Adv-GLP1 (7-36) group, AdV-Ctrl group, and PBS group in pancreatic carcinoma mouse model as shown in Example IV(B).

FIG. 28A shows Lcn2 expression in CD8+ T cells cultured with Panc02 cells with or without the presence of GLP-1(7-36) in pancreatic carcinoma mouse model as shown in Example IV(C).

FIG. 28B shows Lcn2 expression in CD8+ T cells, Panc02 cells cultured with CD8+ T cells, with or without the presence of GLP-1(7-36) and liraglutide in pancreatic carcinoma mouse model as shown in Example IV(C).

FIG. 29 shows that AdV-GLP-1(7-36)-CMV/EF1a (CD8-Panc02-AdV-GLP1(7-36)) significantly inhibited CD8+ T cell-mediated recruitment of neutrophils in vitro compared to control Ad5 treatment (CD8-Panc02-AdV-Ctrl) as shown in Example IV(C).

FIG. 30 shows that recombinant adenovirus expressing a GLP1 (7-37/A8G) exerts oncolytic effect on non-small lung cancer cells.

FIG. 31 shows the experiment design of evaluation of an embodiment of a recombinant adenovirus expressing GLP1(7-37/A8G) in a pancreatic carcinoma mouse model, e.g., Example V(B).

FIG. 32 shows that in a pancreatic carcinoma mouse model as shown in FIG. 31, the tumor growth rate in the group treated with an embodiment of a recombinant expressing GLP1(7-36/A8G) (AdV-GLP-1(7-36/A8G)) was statistically significantly slower compared to the group treated with control adenovirus that does not express a target peptide (AdV-Ctrl) (P<0.0001) and the negative group treated with PBS (PBS), respectively.

FIG. 33 shows that in pancreatic carcinoma mouse model as shown in FIG. 31 (e.g., Example V(B)), the survival rate in the group treated with an embodiment of a recombinant adenovirus expressing GLP1(7-36) (AdV-GLP1(7-36/A8G)) significantly increased compared to the group treated with control adenovirus that does not express a target peptide (AdV-Ctrl), p<0.05.

FIG. 34 shows that in pancreatic carcinoma mouse model as shown in FIG. 31 (e.g., Example V(B)), no significant difference was shown in the body weights among the groups treated with an embodiment of a recombinant adenovirus expressing GLP1(7-36) (AdV-GLP1(7-36/A8G)), control adenovirus that does not express a target peptide (AdV-Ctrl), and the negative group treated with PBS (PBS), respectively.

FIG. 35 shows the experiment design of evaluation of an embodiment of a recombinant adenovirus expressing GLP1(7-36)-PLGF2 or PLGF2-GLP1(7-36) in pancreatic carcinoma mouse model, e.g., Example VI.

FIG. 36A shows the tumor growth data of negative control group treated with PBS in pancreatic carcinoma mouse model as shown in FIG. 35 (e.g., Example VI).

FIG. 36B shows the tumor growth data of the AdV-GLP1 group treated with an embodiment of a recombinant adenovirus expressing GLP1(7-36)-PLGF2 in pancreatic carcinoma mouse model as shown in FIG. 35 (e.g., Example VI).

FIG. 36C shows the tumor growth data of the AdV-GLP1 group treated with an embodiment of a recombinant adenovirus expressing PLGF2-GLP1(7-36) in pancreatic carcinoma mouse model as shown in FIG. 35 (e.g., Example VI).

### DETAILED DESCRIPTION

Glucagon-like peptide-1 (GLP1), a polypeptide consisting of 30 amino acid residues, is an agonist of glucagon-like peptide-1 receptor that not only lowers blood glucose but also significantly inhibits multiple tumor-promoting inflammatory pathways, including the activation of NF-κB/IL-6/STAT3 pathway, the production of NLRP3/IL-1β inflammasome, and the like.

Provided herein are recombinant adenovirus (e.g., adenovirus subtype C, Ad5) or adenoviral vector comprising a first promoter, an EIA early activation replication element, an optional second promoter, and a nucleotide (e.g., DNA) sequence encoding a target protein, the target protein comprising a glucagon-like peptide-1 (GLP-1) receptor agonist, the first promoter being a constitutive promoter, and the optional second promoter being a constitutive promoter which may be the same as or different from the first promoter; and their preparation and uses.

As shown in the Examples section, several embodiments of Ad5 adenovirus or adenoviral vector comprising a nucleotide (e.g., DNA) sequence encoding a target protein comprising a GLP-1 receptor agonist were prepared with one or two promoters upstream of the nucleotide (e.g., DNA), which expressed the target protein in vivo and in vitro and showed not only tumor growth inhibition in vitro and in vivo but also some tumor recession effects in vivo for various tumors (e.g., non-small lung cancer, pancreatic cancer, colon cancer, melanoma, glioma, and kidney cancer) without significant side effects observed.

Furthermore, at least in one example of Ad5 adenovirus or adenoviral vector comprising a nucleotide (e.g., DNA) sequence encoding a target protein comprising GLP-1(7-37) induced CD8+T in pancreatic carcinoma instead of nature killer (NK) cell-mediated antitumor effect in hepatocellular carcinoma induced by a GLP-1 analogue Liraglutide administered intratumorally to animal models (Xian Lu, Chun Xu, Jie Dong, Shuguang Zuo, Hailin Zhang, Chunping Jiang, Junhua Wu, Jiwu Wei, Liraglutide activates nature killer cell-mediated antitumor responses by inhibiting IL-6/STAT3 signaling in hepatocellular carcinoma, Translational Oncology, Volume 14, Issue 1, 2021, 100872).

### I. RECOMBINANT ADENOVIRUS OR ADENOVIRAL VECTOR, AND PHARMACEUTICAL COMPOSITIONS COMPRISING SAME

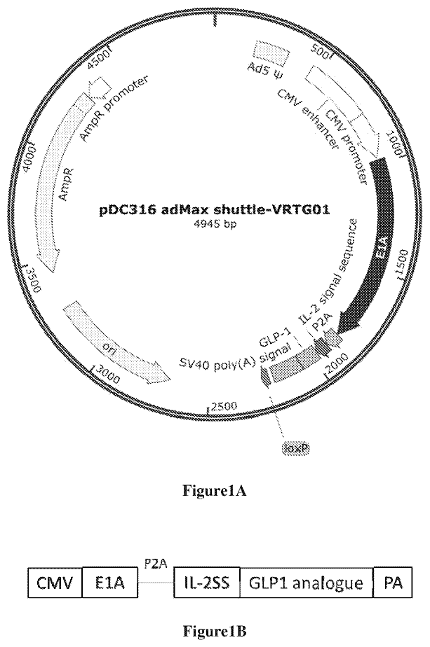

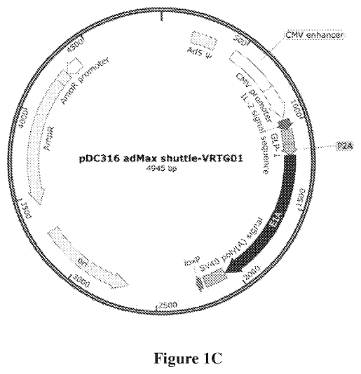

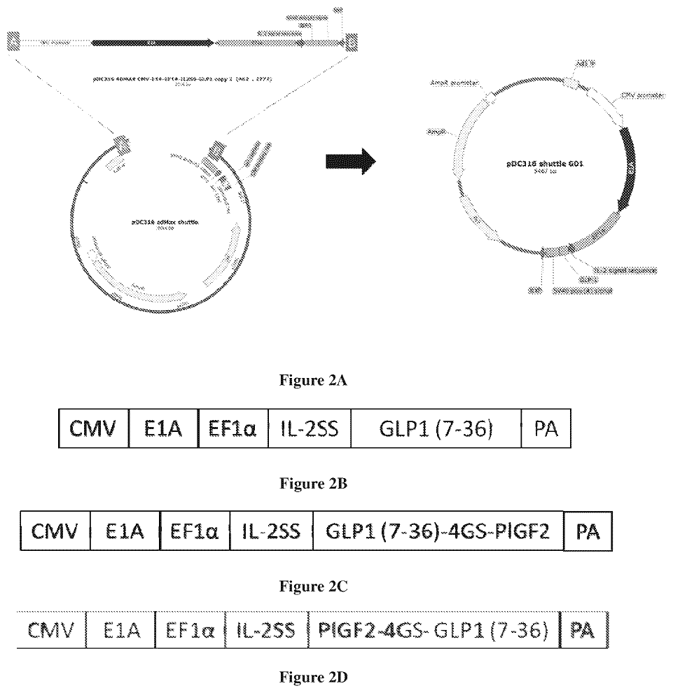

One aspect of the disclosure relates to a recombinant adenovirus or adenoviral vector comprising a first promoter, an E1A early activation replication element, an optional second promoter, and a nucleotide (e.g., DNA) sequence encoding a target protein, the target protein comprising a glucagon-like peptide-1 (GLP-1) receptor agonist, the first promoter being a constitutive promoter, and the optional second promoter being a constitutive promoter which may be the same as or different from the first promoter. In certain embodiments, the nucleotide (e.g., DNA) sequence further comprises a DNA sequence encoding a IL-2 signal peptide facilitate secretion of the target peptide extracellularly. In certain embodiments, when the target protein is GLP-1(7-36), the recombinant adenovirus or adenoviral vector comprises two promoters. In certain embodiments, the E1A early activation replication element may be upstream of the nucleotide (e.g., DNA) sequence or downstream of the nucleotide (e.g., DNA) sequence, as long as both the EIA early activation replication element and the nucleotide (e.g., DNA) sequence are between the first promoter and poly(A) tail. See, e.g., FIGS. 1A, 1C, 2A and 2E, wherein the first promoter is CMV as an example.

Examples of the recombinant adenovirus include, without limitation, adenovirus subtype C, Ad5, etc.

Certain embodiments of the recombinant adenovirus (e.g., Ad5) or adenoviral vector disclosed herein may be useful for treating or preventing one or more tumors in a subject. In certain embodiments, the recombinant adenovirus (e.g., Ad5) or adenoviral vector disclosed herein induces CD8+T cell-mediated antitumor effects in the subject. In certain embodiments, the recombinant adenovirus (e.g., Ad5) or adenoviral vector disclosed herein causes tumor recession in the subject.

Certain embodiments of the recombinant adenovirus (e.g., Ad5) or adenoviral vector disclosed herein comprises one or more constitutive promoters selected from the group consisting of CMV promoter and EF1a promoter. The plurality of constitutive promoters may be the same or different. For example, the recombinant adenovirus (e.g., Ad5) or adenoviral vector may comprise CMV promoter as both the first and second promoters; the recombinant adenovirus (e.g., Ad5) or adenoviral vector may comprise EF1a promoter as both the first and second promoters; the recombinant adenovirus (e.g., Ad5) or adenoviral vector may comprise CMV promoter as the first promoter and EF1a promoter as the second promoter; and alternatively, the recombinant adenovirus (e.g., Ad5) or adenoviral vector may comprise EF1a promoter as the first promoter and CMV promoter as the second promoter.

Certain embodiments of the recombinant adenovirus (e.g., Ad5) or adenoviral vector disclosed herein comprises two constitutive promoters, and a DNA sequence encoding GLP-1(7-36), GLP-1(7-36) having an amino acid sequence of SEQ ID NO:1.

Examples of the GLP-1 receptor agonist include, without limitation, an amino acid sequence with 80% homology or higher to GLP-1, a bioactive fragment of GLP-1 (e.g., GLP-1(7-36) (SEQ ID NO.: 1), GLP-1(7-37)), or a GLP-1 analog, e.g., with one or more mutants such as GLP-1(7-37/A8G) (SEQ ID No.: 2). Certain embodiments of the recombinant adenovirus (e.g., Ad5) or adenoviral vector disclosed herein comprises a secretory glucagon-like peptide-1 protein as the GLP-1 receptor agonist. Examples of GLP-1 analogs include, without limitation, GLP-1 fragments with one or more substituted amino acids, one or more omitted amino acids, and/or one or more additional amino acids, while the GLP-1 analogs are GLP-1 receptor agonists.

In certain embodiments, the target protein comprises a fusion protein of a tumor homing protein (e.g., PLGF2) and the GLP-1 receptor agonist. In certain embodiments, the fusion protein further comprises a linker (e.g., a 4GS linker such as (GGGGS)ₙ, n=1, 2, 3, 4, 5, 6, 7, 8, 9, or 10) linking the tumor homing protein (e.g., PLGF2) and the GLP-1 receptor agonist. In certain embodiments, the GLP-1 receptor agonist of the fusion protein is closer to the N-terminal than the tumor homing protein (e.g., PLGF2). In certain embodiments, the tumor homing protein (e.g., PLGF2) of the fusion protein is closer to the N-terminal than the GLP-1 receptor agonist.

Examples of the fusion proteins include, without limitation, GLP-1(7-36)-4G-PLGF2 (e.g., SEQ ID NO.: 3, wherein n=3 for the 4GS linker, Table 1), PLGF2-4GS-GLP-1(7-36) (e.g., SEQ ID NO.: 4, wherein n=3 for the 4GS linker, Table 1), GLP-1(7-37)-4GS-PLGF2, PLGF2-4GS-GLP-1(7-37), GLP-1(7-37/A8G)-4GS-PLGF2, and PLGF2-4GS-GLP-1(7-37/A8G).

Certain embodiments of the recombinant adenovirus (e.g., Ad5) or adenoviral vector disclosed herein comprises one or two constitutive promoters, and a DNA sequence encoding the target protein, as summarized in Table 1 (FIGS. 1A-2D).

TABLE 1
Examples of target proteins and DNA sequences encoding same

| SEQ Name | | |
|---|---|---|
| and | SEQ ID | |
| description | NO. | Sequence |
| GLP1 | 1 | HAEGTFTSDVSSYLEGQAAKEFIAWLVKGR |
| (7-36) | | |
| GLP-1 | 2 | HGEGTFTSDVSSYLEGQAAKEFIAWLVKGRG |
| (7-37/A8G) | | |
| GLP-1 | 3 | HAEGTFTSDVSSYLEGQAAKEFIAWLVKGRGGGGSGGGGS |
| (7-36)-4GS- | | GGGGSRRPKGRGKRRRENQRPTDCHL |
| PLGF2 | | |
| PLGF2- | 4 | RRPKGRGKRRRENQRPTDCHLGGGGSGGGGSGGGGSHAE |
| 4GS-GLP- | | GTFTSDVSSYLEGQAAKEFIAWLVKGR |
| 1(7-36) | | |
| DNA | 5 | CACGCCGAGGGCACATTTACAAGCGACGTGTCCAGCTAC |
| sequence | | CTGGAAGGCCAGGCCGCTAAAGAGTTCATCGCCTGGCTG |
| encoding | | GTGCGGGGAAGGGGC |
| GLP-1 | | |
| (7-36) | | |
| DNA | 6 | CATGGTGAAGGGACCTTTACCAGTGATGTAAGTTCTTAT |
| sequence | | TTGGAAGGCCAAGCTGCCAAGGAATTCATTGCTTGGCTG |
| encoding | | GTGAAAGGCCGAGGA |
| GLP-1 | | |
| (7-37/A8G) | | |
| DNA | 7 | CATGCTGAAGGGACCTTTACCAGTGATGTAAGTTCTTAT |
| sequence | | TTGGAAGGCCAAGCTGCCAAGGAATTCATTGCTTGGCTG |
| encoding | | GTGAAAGGCCGAGGTGGAGGCGGTTCAGGCGGAGGTGG |
| GLP-1 | | CTCTGGCGGTGGCGGATCGAggagacccaagggcagggggaagagga |
| (7-36)-4GS- | | ggagagagaaccagagacccacagactgccacctg |
| PLGF2 | | |
| DNA | 8 | Aggagacccaagggcagggggaagaggaggagagagaaccagagacccacagactgc |
| sequence | | cacctgGGTGGAGGCGGTTCAGGCGGAGGTGGCTCTGGCGG |
| encoding | | TGGCGGATCGCATGCTGAAGGGACCTTTACCAGTGATGT |
| PLGF2- | | AAGTTCTTATTTGGAAGGCCAAGCTGCCAAGGAATTCAT |
| 4GS-GLP- | | TGCTTGGCTGGTGAAAGGCCGA |
| 1(7-36) | | |

Another aspect of the disclosure relates to pharmaceutical composition comprising the recombinant adenovirus (e.g., Ad5) or adenoviral vector disclosed herein, and a pharmaceutically acceptable carrier, adjuvant, additive or combination thereof.

Examples of acceptable carriers include physiologically acceptable solutions, such as sterile saline and sterile buffered saline.

The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

The recombinant adenovirus or adenoviral vector, or pharmaceutical composition comprising a recombinant adenovirus or adenoviral vector described herein may be stored as an aqueous solution or a lyophilized product in a unit or multiple dose container such as a sealed ampoule or a vial.

### II. USES OF THE RECOMBINANT ADENOVIRUS (E.G., AD5) OR ADENOVIRAL VECTOR, AND PHARMACEUTICAL COMPOSITIONS COMPRISING SAME

Another aspect of the disclosure relates to uses of the recombinant adenovirus (e.g., Ad5) or adenoviral vector disclosed herein or pharmaceutical composition comprising same for antitumor treatment and/or prevention in a subject. In certain embodiments, the antitumor treatment and/or prevention comprising inducing CD8+T cell-mediated antitumor effects in the subject.

In certain embodiments, the method of inducing CD8+T cell-mediated antitumor effects in a subject comprising administering to the subject a therapeutically effective amount of the recombinant adenovirus (e.g., Ad5) or adenoviral vector or the pharmaceutical composition of same.

In certain embodiments, the method of treating one or more cancers in a subject comprising administering to the subject a therapeutically effective amount of the recombinant adenovirus (e.g., Ad5) or adenoviral vector or the pharmaceutical composition of same.

Examples of the one or more cancers include, without limitation, liver cancer, pancreatic cancer, colon cancer, glioma, lung cancer, esophageal carcinoma, gastric cancer, breast cancer, ovarian cancer, prostate cancer, kidney cancer, squamous cell carcinoma of head and neck, melanoma, multiple myeloma, and lymphoma. In certain embodiments, the lung cancer is non-small lung cancer.

In certain embodiments, the cancer is a metastasis.

Certain embodiments of the antitumor methods disclosed herein further comprise inhibiting tumor invasion and/or metastasis.

Certain embodiments of the antitumor methods disclosed herein further comprise one or more effects selected from the group consisting of: reducing expression of LCN2 in CD8+T cells, and reducing intratumoral infiltration of neutrophils.

Certain embodiments of the antitumor methods disclosed herein further comprise delaying a cachexia progress of the subject.

Certain embodiments of the antitumor methods disclosed herein further comprise prolonging life span of the subject.

The term “effective amount” as used herein refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing or treating a target condition (e.g., one or more tumors), alleviating symptoms associated with the condition, or producing a desired physiological effect. In such a case, the effective amount of a compound is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and pharmacological arts will be able to determine an effective amount or therapeutically effective amount through routine experimentation, namely by monitoring a cell's or subject's response to administration of a compound and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

In some embodiments, the recombinant adenovirus (e.g., Ad5) or adenoviral vector disclosed herein or pharmaceutical composition disclosed herein may be used in combination with other known pharmaceutical products, such as immune response-promoting peptides and antibacterial agents (synthetic antibacterial agents). The recombinant adenovirus (e.g., Ad5) or adenoviral vector disclosed herein or pharmaceutical composition disclosed herein may further comprise other drugs and additives. Examples of drugs or additives that may be used in conjunction with a recombinant adenovirus (e.g., Ad5) or adenoviral vector disclosed herein or pharmaceutical composition disclosed herein include drugs that aid intracellular uptake of the recombinant adenovirus (e.g., Ad5) or adenoviral vector or pharmaceutical composition disclosed herein, liposome and other drugs and/or additives that facilitate transfection, (e.g., fluorocarbon emulsifiers, cochleates, tubules, golden particles, biodegradable microspheres, and cationic polymers).

In some embodiments, the amount of the active ingredient contained in the recombinant adenovirus (e.g., Ad5) or adenoviral vector or pharmaceutical composition disclosed herein may be selected from a wide range of concentrations, ifu, weight to volume percent (w/v %) or other quantitative measure of active ingredient amount, as long as it is a therapeutically or pharmaceutically effective amount. The dosage of the recombinant adenovirus (e.g., Ad5) or adenoviral vector or pharmaceutical composition disclosed herein may be appropriately selected from a wide range according to the desired therapeutic effect, the administration method (administration route), the therapeutic period, the patient's age, gender, and other conditions, etc.

In some aspects, when a recombinant adenovirus (e.g., Ad5) or adenoviral vector is administered to a human subject as an active ingredient, the dosage of the recombinant adenovirus (e.g., Ad5) or adenoviral vector may be administered in an amount approximately corresponding to about 5×10E5 to about 5×10E10 ifu, about 5×10E6 to about 5×10E9 ifu, about 1×10E7 to about 1×10E9 ifu, or about 5×10E7 to about 5×10E8 ifu, about 1×10E8 to about 1×10E10 ifu, about 5×10E8 to about 5×10E10 ifu, about 5×10E7 to about 5×10E11, about 1×10E8 to about 1×10E10, or about 1×10E8 to about 1×10E11, per subject.

In further aspects, when a recombinant adenovirus (e.g., Ad5) or adenoviral vector is administered to a subject as an active ingredient, the dosage may be selected from a wide range in terms of the amount of expressible DNA introduced into the recombinant adenovirus (e.g., Ad5) or adenoviral vector. The dosage also depends on the strength of the transcription and translation promoters used in any transfer vectors used.

In some embodiments, the recombinant adenovirus (e.g., Ad5) or adenoviral vector or pharmaceutical composition described herein may be administered by directly injecting a recombinant adenovirus (e.g., Ad5) or adenoviral vector suspension prepared by suspending the recombinant adenovirus (e.g., Ad5) or adenoviral vector in PBS (phosphate buffered saline) or saline into a target site (e.g., the one or more cancers). The recombinant adenovirus (e.g., Ad5) or adenoviral vector or pharmaceutical composition disclosed herein may be administered more than once. More specifically, after the initial administration, one or more additional recombinant adenovirus (e.g., Ad5) or adenoviral vector may be administered.

In certain embodiments of the antitumor methods disclosed herein, the recombinant adenovirus (e.g., Ad5) or adenoviral vector or the pharmaceutical composition comprising same is administered as a single-drug therapy, adjuvant therapy, or combination therapy.

Certain embodiments of the antitumor methods disclosed herein, further comprise one or more therapies selected from the group consisting of one or more chemotherapies, and one or more radiotherapies.

In certain embodiments, the therapeutically effective amount of the recombinant adenovirus (e.g., Ad5) or adenoviral vector or the pharmaceutical composition comprising same is administered once a day.

In certain embodiments, the therapeutically effective amount of the recombinant adenovirus (e.g., Ad5) or adenoviral vector or the pharmaceutical composition comprising same is administered once every other day.

In certain embodiments, the therapeutically effective amount of the recombinant adenovirus (e.g., Ad5) or adenoviral vector or the pharmaceutical composition comprising same is administered for at least about one week, at least about two weeks, at least about three weeks, at least about four weeks, at least about one month, at least about two months, at least about three months, at least about four months, at least about five months, at least about six months, at least about seven months, at least about eight months, at least about nine months, at least about ten months, at least about eleven months, at least about twelve months, at least about one year, at least about two years, at least about three years, at least about four years, or at least about five years. In certain embodiments, the therapeutically effective amount of the recombinant adenovirus (e.g., Ad5) or adenoviral vector or the pharmaceutical composition comprising same is administered to one or more cancers in a subject until the one or more cancers substantially recess or disappear.

### III. PREPARATIONS OF THE RECOMBINANT ADENOVIRUS (E.G., AD5) OR ADENOVIRAL VECTOR, AND PHARMACEUTICAL COMPOSITIONS COMPRISING SAME

Another aspect of the disclosure relates to preparation of the recombinant adenovirus (e.g., Ad5) or adenoviral vector. One embodiment of the preparation of an embodiment of the recombinant adenovirus (e.g., Ad5) or adenoviral vector comprises i) shuttle plasmid construction; ii) virus rescue; and iii) virus amplification as disclosed herein.

In one embodiment, the shuttle plasmid construction step comprises preparing a shuttle plasmid comprising a nucleotide (e.g., DNA) encoding a target protein, the target protein comprising a GLP-1 receptor agonist as disclosed herein, e.g., without limitation, GLP-1, GLP-1 fragments (e.g., GLP-1(7-36), GLP-1(7-37)), and GLP-1 analogs (e.g., GLP-1 fragments with one or more substituted amino acids, one or more omitted amino acids, and/or one or more additional amino acids). In certain embodiments, the target protein comprises a fusion protein of a GLP-1 receptor agonist and another protein (e.g., PLGF2).

The following examples are intended to illustrate various embodiments of the invention. As such, the specific embodiments discussed are not to be construed as limitations on the scope of the invention. It will be apparent to one skilled int eh art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### IV. Examples

The following examples illustrate that Ad5 Examples are summarized below.

TABLE 2
Embodiments of the recombinant Ad5 adenovirus

| | DNA | | | | Shuttle | | | |
|---|---|---|---|---|---|---|---|---|
| Embodiment | sequence | | | | plasmid | | | |
| of the | encoding | | | | incorporated | Ad5- | | |
| recombinant | the | | The | The | with | target | | Anti- |
| Ad5 | target | Target | first | second | the target | protein | Expression | tumor |
| adenovirus | protein | protein | promoter | promoter | DNA | preparation | evaluation | effects |
| AdV- | SEQ | SEQ | CMV | EF1a | pAdV- | Example | Example | Examples |
| GLP-1(7- | ID | ID | | | GLP-1(7- | I | II | III, IV |
| 36)- | NO: 5 | NO: 1 | | | 36)- | | | |
| CMV/EF1a | | | | | CMV/EF1a | | | |
| AdV- | SEQ | SEQ | CMV | N/A | pAdV- | Example | Example | Example |
| GLP-1(7- | ID | ID | | | GLP-1(7- | I | II | V |
| 37/A8G)- | NO: 6 | NO: 2 | | | A8G)- | | | |
| CMV | | | | | CMV | | | |
| AdV- | SEQ | SEQ | CMV | EF1a | pAdV- | Example | Example | Example |
| GLP-1(7- | ID | ID | | | GLP-1(7- | I | II | VI |
| 36)-4GS- | NO: 7 | NO: 3 | | | 36)-4GS- | | | |
| PLGF2- | | | | | PLGF2- | | | |
| CMV/EF1a | | | | | CMV/EF1a | | | |
| AdV- | SEQ | SEQ | CMV | EF1a | pAdV- | Example | Example | Example |
| PLGF2- | ID | ID | | | PLGF2- | I | II | VI |
| 4GS- | NO: 8 | NO: 4 | | | 4GS- | | | |
| GLP-1(7- | | | | | GLP-1(7- | | | |
| 36)- | | | | | 36)- | | | |
| CMV/EF1a | | | | | CMV/EF1a | | | |

### Example I. Preparation of Embodiments of the Recombinant Ad5 Adenovirus or Adenoviral Vectors with Single Promoter or Two Promoters

Embodiments of the recombinant Ad5 adenovirus or adenoviral vectors with single promoter were prepared via three steps: i) Ad5 shuttle plasmid construction; ii) Ad5 virus rescue; and iii) virus amplification as described below.

### I(A). Ad5 Shuttle Plasmid Construction

DNA sequences encoding the desired elements (e.g., Homologous A-CMV-E1A-P2A-GLP1(7-36)-Homologous B for an embodiment of recombinant adenovirus with CMV as the single promoter, FIGS. 1A-1B; Homologous A-CMV-EIA-EF1a-GLP1(7-36)-Homologous B for an embodiment of recombinant adenovirus with CMV as the first promoter and EF1a as the second promoter, FIGS. 2A-2D) were synthesized in vitro. Shuttle plasmid (e.g., pDC316 AdMax shuttle) was cleaved with two restriction enzymes (e.g., XbaI and HindIII) and infused with the synthetic DNA sequence in the presence of In-Fusion enzyme. The shuttle plasmid comprising the DNA sequence encoding the desired target protein, i.e., the target DNA (see examples in Table 1), was confirmed by DNA sequencing.

### I(B). Ad5 Virus Rescue

293T cells were transfected with pBHG-lox-(delta)E1E3-Cre and the Ad5 shuttle plasmid incorporated with the target DNA in a T25 flask and cultured at 37° C. and 5% CO₂. 7-10 days later, the cells were blown and collected into a 15 mL centrifuge tube using a 1 mL culture medium, repeatedly freeze-thawed 2 times, and centrifuged at 3,000 rpm/min for 15 min. The virus supernatant was collected and stored at −80° C. as the viral stock.

### I(C). Ad5 Virus Amplification

50 μL of viral stock solution was added to a 10 cm dish containing 60% 293T cells and cultured at 37° C. and 5% CO₂. The cells were harvested when display cytopathic effect.—The viruses were collected according to the above method and purified using cesium chloride density-gradient centrifugation. Titer assay was performed using the TCID50 method.

### Example II. Viral Function Evaluation of Embodiments of Embodiments of the Recombinant Ad5 Adenovirus

### II(A). Expression and Secretion Function of GLP1

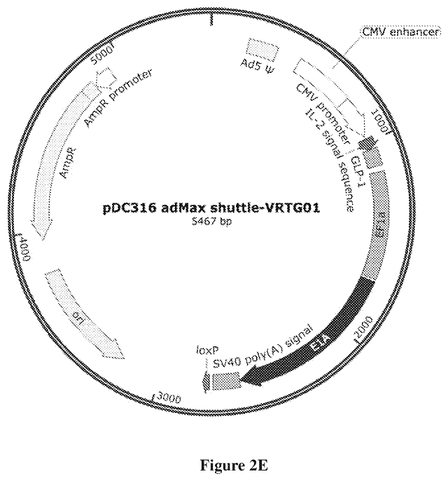

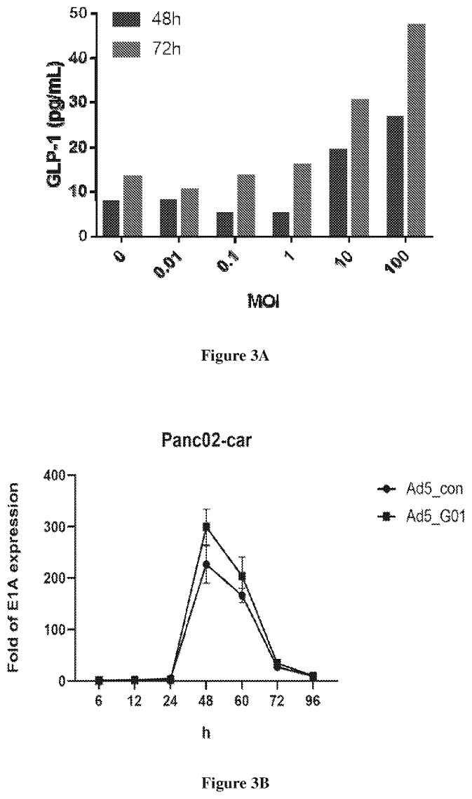

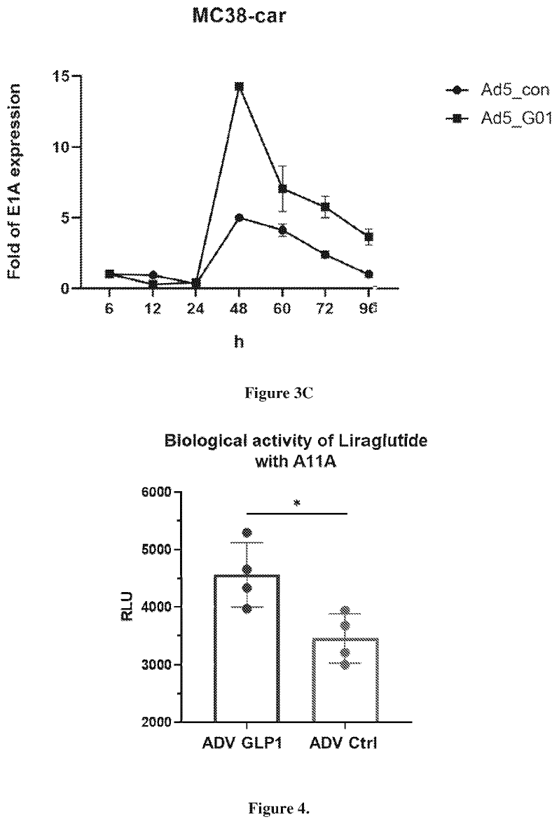

A549 cells were placed in a six-well plate (8×10E5/well) and incubated overnight in DMEM in the presence of 10% FBS; the medium was removed and DMEM medium containing 2% FBS (1 mL/well) was added; embodiments of the recombinant Ad5 adenovirus incorporated with the target DNA (GLP-1(7-36))(Example I, MOI=100, 10, 1, 0.1, 0.01 or 0) were added and supernatant was collected after 48 h or 72 h of the virus treatments; and the expression and secretion function of GLP1 were evaluated by ELISA (FIG. 3A, the left column for each MOI showed GLP-1(7-36) concentration of the sample collected after 48 h of virus treatment; and the right column showed GLP-1(7-36) concentration of the sample collected after 72 h of virus treatment).

The tumor cells (Panc02 and MC38) were infected with embodiments of the recombinant Ad5 virus incorporated with the target DNA (Example I) and Ad5 containing only the E1A sequence as the viral replication element without the target DNA sequence (Ad5 con) viruses with the same MOI. Cells were collected at different time points. Virus suspensions were obtained after repeated freeze-thaw and centrifugation. Viral titer assay was performed using 293T cells to analyze the changes in viral replication capacity (FIGS. 3B-3C). Viral replication capacity in pancreatic and colorectal carcinoma cells of one embodiment of the recombinant adenovirus expressing GLP1 (Ad5_G01, FIGS. 3B-3C) was higher than control adenovirus that did not express GLP1 (Ad5_con, FIGS. 3B-3C).

II(B). A11A Cell Line Treated with Embodiments of the Recombinant Ad5 Adenovirus Showed Comparable Biological Activity of the Target Proteins and Liraglutide:

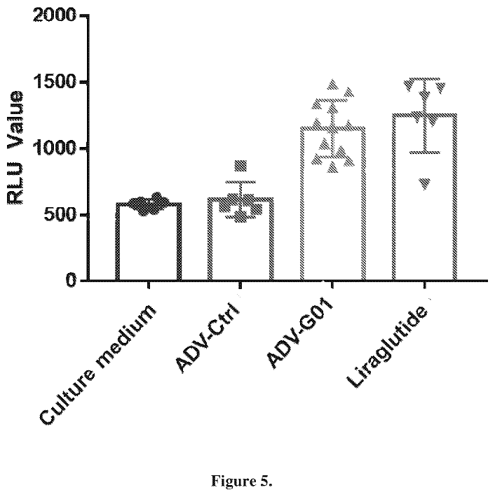

A11A is a cell line stably expressing a fusion report gene, which was used for monitoring the bioactivity of GLP-1. HEK293 cells (4.7E+05˜5.2E+05/well) were added in a six-well plate and incubated 37 C and 5% CO₂ over night; embodiments of the recombinant Ad5 adenovirus incorporated with target DNA encoding target proteins (e.g., AdV-GLP-1(7-37/A8G) and AdV-GLP-1(7-36)-CMV/EF1a prepared according to Example 1) were added at a desired ifu (e.g., MOI=15), and collect cells and all liquid in the wells respectively, 48-72 h after virus infection and stored at −70 C as the GLP-1 receptor agonist samples for A11A cell treatment, negative control samples were prepared the same except that Ad5 con was added instead of the recombinant Ad5 adenovirus AdV-GLP-1(7-37/A8G) and AdV-GLP-1(7-36)-CMV/EF1a. A11A cells were added to a 96-well plate (5×10E3 cell/well) and incubated overnight; 100 μL of the GLP-1 receptor agonist samples, the negative control samples, or liraglutide (1,000 nM, 100 μL) were added to the A11A cells, respectively; and fluorescent intensities were measured after 4 hrs of treatment to show bioactivity of GLP-1 agonists in the GLP-1 agonist samples (Table 3, and FIGS. 4-5).

TABLE 3
AdV-GLP-1(7-37/A8G)-CMV A11A data

| | AdV-GLP-1(7-37/A8G)-CMV | Ad5 con |
|---|---|---|
| RLU | 4332 | 3937 |
| | 4645 | 3673 |
| | 3972 | 3003 |
| | 5293 | 3216 |
| Ave. RLU | 4560.5 | 3457.25 |

### Example III. In Vivo Anti-Tumor Effects of the Embodiment of the Recombinant Ad5 Adenovirus AdV-GLP-1(7-36)-CMV/EF1a

AdV-GLP-1(7-36)-CMV/EF1a was prepared according to the method of Example I(B) with the target protein having a sequence of SEQ ID NO.: 1, a target DNA having a sequence of SEQ ID NO.: 5. The viral replication, expression of GLP-1 receptor agonist, and biological activity of the GLP-1 receptor agonist expressed were validated according to the method of Example II (see, e.g., FIG. 5).

### III(A). In Vivo Anti-Tumor Effects of AdV-GLP-1(7-36)-CMV/EF1a on Pancreatic Cancer Animal Models

III(A)(i) Panc02-Car s.c. Animal Model

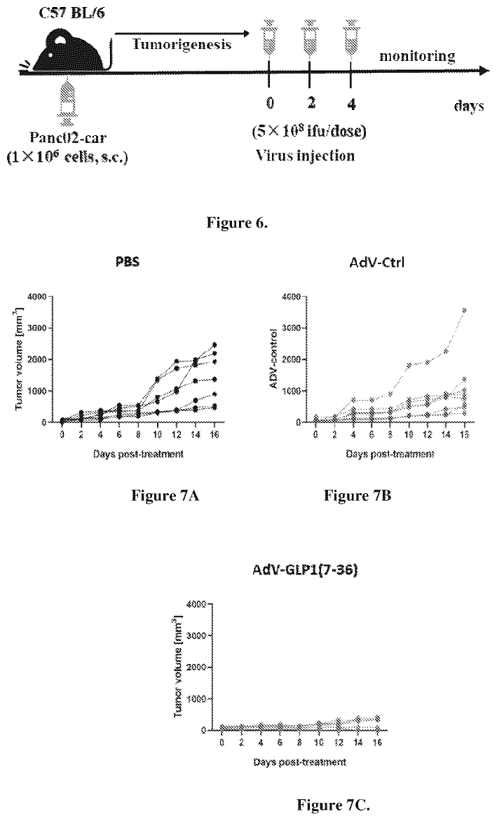

6-8 weeks old C57BL/6 mice were selected to establish a subcutaneous tumor model with panc02-car cells suspended in 10% matrigel in PBS (1×10E6 cells/mice, 18 mice total, FIG. 6), and randomly divided into 3 groups after the tumor size reached about 100 mm³: a negative control group treated with PBS only (PBS, 0.1 mL), a control Ad5 group treated with Ad5 control (AdV-Ctrl, 5×10E8 ifu/dose), and a treatment group treated with Ad5 incorporated with the target DNA AdV-GLP-1(7-36)-CMV/EF1a (AdV-GLP1(7-36), 5×10E8 ifu/dose), all treatments were injected intratumorally every other day for three doses (FIG. 6). The tumor volume and body weight were measured and the survival period was recorded after natural death of the mice.

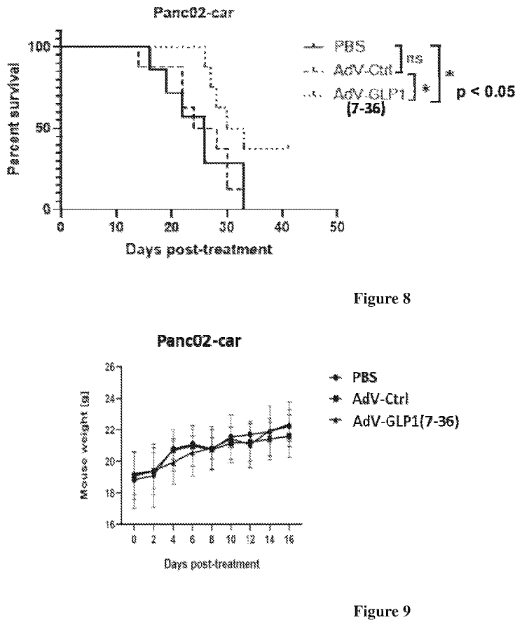

AdV-GLP-1(7-36)-CMV/EF1a treatment showed slower tumor growth rate (FIG. 7C) than the control Ad5 (p<0.0001, FIG. 7B) and the negative control group treated with PBS only (p<0.0001, FIG. 7A); and statistically significantly longer survival rate compared to both the control Ad5 group and the negative control group (p<0.05, FIG. 8). No significant change of body weights was observed among the three treatment groups (FIG. 9).

### V(A)(ii) Orthotopic Pancreatic Carcinoma Model

The tumor was grown in the mouse pancreas. The data showed in PPT 7/18/S. 8 was subcutaneous pancreatic carcinoma model. They are the same tumor type but not the same model. Since the orthotopic tumor on pancreas could not be measured by calliper as the subcutaneous tumor. We have to monitor the tumor growth by luminescence. The significance was indicated in the figure by *. p<0.05

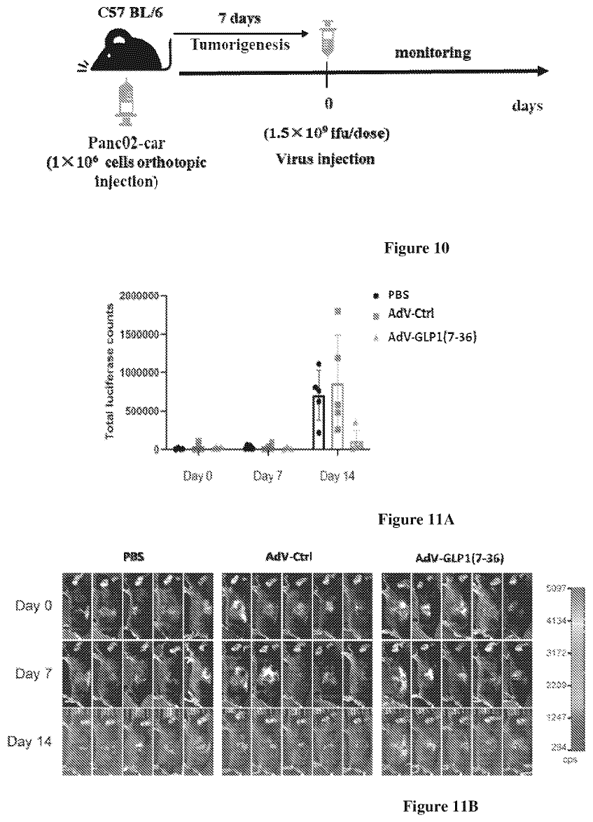

6-8 weeks old C57BL/6 mice were selected to establish an orthotopic pancreatic carcinoma model on pancreas with panc02-car cells suspended in 10% matrigel in PBS (1×10E6 cells/mice, 18 mice total, FIG. 10), and randomly divided into 3 groups after the tumor size reached about 100 mm³: A negative control group treated with PBS only (PBS, 0.1 mL), a control Ad5 group treated with Ad5 control (AdV-Ctrl, 1.5×10E9 ifu/dose), and a treatment group treated with Ad5 incorporated with the target DNA AdV-GLP-1(7-36)-CMV/EF1a (AdV-GLP1(7-36), 1.5×10E9 ifu/dose), all treatments were injected intratumorally for one dose (FIG. 10). The tumor volume and body weight were measured by luminescence and the survival period was recorded after natural death of the mice.

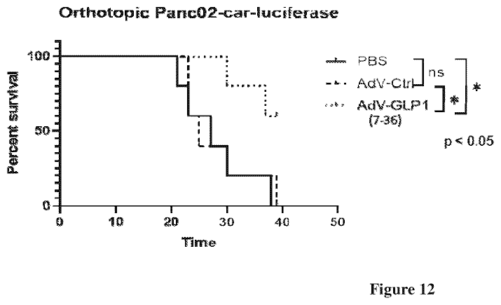

AdV-GLP-1(7-36)-CMV/EF1a treatment showed statistically significantly slower tumor growth rate (AdV-GLP-1(7-36), FIGS. 11A-11B) than the control Ad5 (p=0.0032, AdV-Ctrl, FIGS. 11A-11B) and the negative control group treated with PBS only (p=0.0002, PBS, FIGS. 11A-11B); and statistically significantly longer survival rate compared to both the control Ad5 group and the negative control group (p<0.05, FIG. 12).

### II(B). In Vivo Anti-Tumor Effects of AdV-GLP-1(7-36)-CMV/EF1a on Colorectal Carcinoma Animal Models

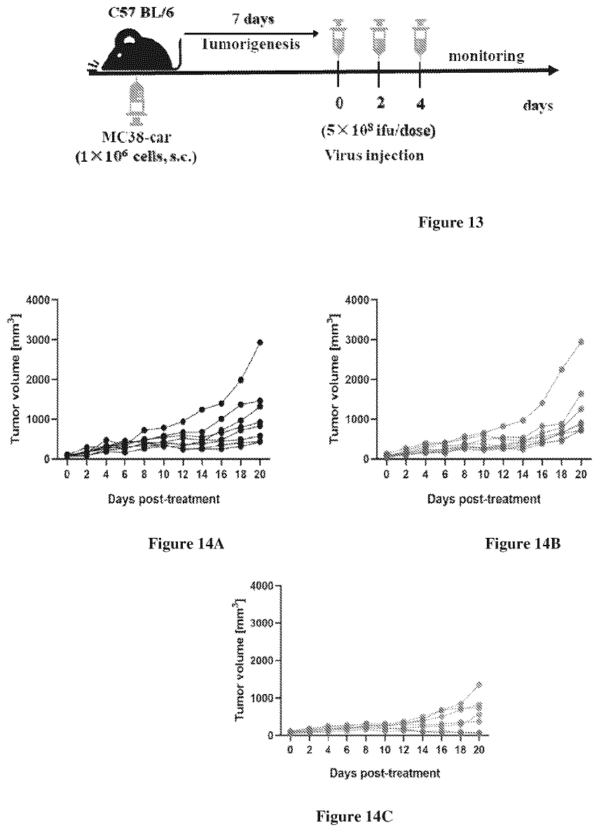

6-8 weeks old C57BL/6 mice were selected to establish a subcutaneous tumor model at the right flank with MC38-car cells suspended in 10% matrigel in PBS (1×10E6 cells/mice, 21 mice total, FIG. 13), and randomly divided into 3 groups after the tumor size reached about 100 mm³: A negative control group treated with PBS only (PBS, 0.1 mL), a control Ad5 group treated with Ad5 control (AdV-Ctrl, 5×10E8 ifu/dose), and a treatment group treated with Ad5 incorporated with the target DNA AdV-GLP-1(7-36)-CMV/EF1a (AdV-GLP1(7-36), 5×10E8 ifu/dose), all treatments were injected intratumorally one dose every other day for three doses (FIG. 13). The tumor volume and body weight were measured and the survival period was recorded after natural death of the mice.

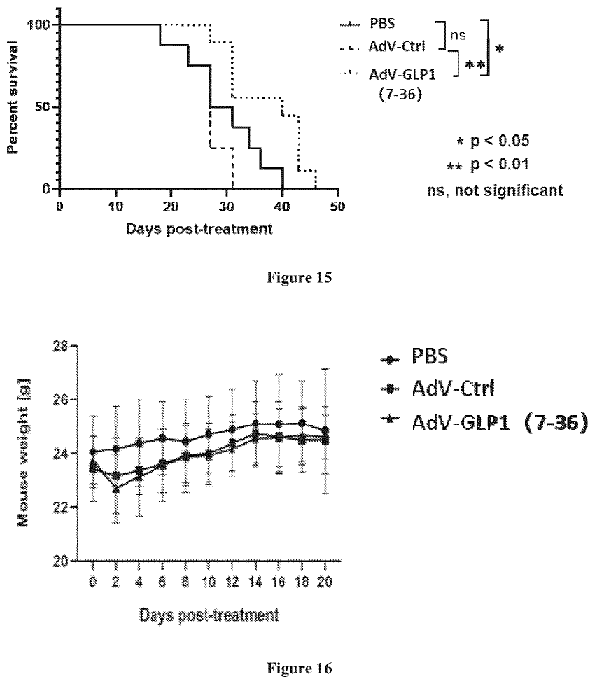

AdV-GLP-1(7-36)-CMV/EF1a treatment showed statistically significantly slower tumor growth rate (FIG. 14C) than the control Ad5 (p<0.0001, FIG. 14B) and the negative control group treated with PBS only (Pp<0.0001, FIG. 14A); and statistically significantly longer survival rate compared to both the control Ad5 group (**: p<0.01) and the negative control group (*: p<0.05, FIG. 15). No significant change of body weights was observed among the three treatment groups (FIG. 16).

### III(C). In Vivo Anti-Tumor Effects of AdV-GLP-1(7-36)-CMV/EF1a on Melanoma Animal Models

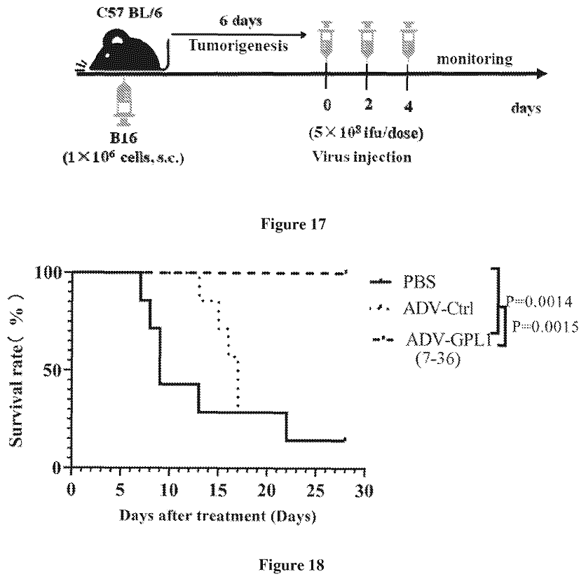

6-8 weeks old C57BL/6 mice were selected to establish a subcutaneous tumor model with B16 cells suspended in 10% matrigel in PBS (1×10E6 cells/mice, 21 mice total, FIG. 17), and randomly divided into 3 groups after the tumor size reached about 100 mm³: A negative control group treated with PBS only (PBS, 0.1 mL), a control Ad5 group treated with Ad5 control (AdV-Ctrl, 5×10E8 ifu/dose), and a treatment group treated with Ad5 incorporated with the target DNA AdV-GLP-1(7-36)-CMV/EF1a (AdV-GLP1(7-36), 5×10E8 ifu/dose), all treatments were injected intratumorally one dose every other day for three doses (FIG. 17). The survival period was recorded after natural death of the mice (FIG. 18).

### II(D). In Vivo Anti-Tumor Effects of AdV-GLP-1(7-36)-CMV/EF1a on Glioma Animal Models

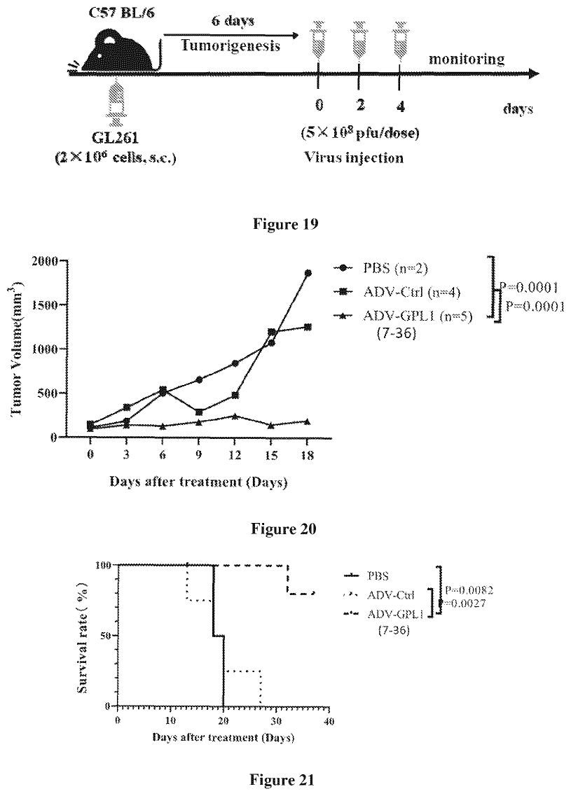

6-8 weeks old C57BL/6 mice were used to establish a subcutaneous tumor model with GL261 glioma cells suspended in 10% matrigel in PBS (1×10E6 cells/mice, 11 mice total, FIG. 19), and randomly divided into 3 groups after the tumor size reached about 100 mm³: a negative control group treated with PBS only (PBS, 0.1 mL, n=2), a control Ad5 group treated with Ad5 control (AdV-Ctrl, 5×10E8 pfu/dose, n=4), and a treatment group treated with Ad5 incorporated with the target DNA AdV-GLP-1(7-36)-CMV/EF1a (AdV-GLP1(7-36), 5×10E8 pfu/dose, n=5), all treatments were injected intratumorally one dose every other day for three doses (FIG. 19). The tumor volumes were measured and the survival period was recorded after natural death of the mice.

AdV-GLP-1(7-36)-CMV/EF1a treatment showed statistically significantly slower tumor growth rate (AdV-GLP1(7-36), FIG. 20) than the control Ad5 (p=0.0001, AdV-Ctrl, FIG. 20) and the negative control group treated with PBS only (p=0.0001, PBS, FIG. 20); and statistically significantly longer survival rate compared to both the control Ad5 group (p=0.0027) and the negative control group (p=0.0082, PBS, FIG. 21).

### II(E). In Vivo Anti-Tumor Effects of AdV-GLP-1(7-36)-CMV/EF1a on Kidney Animal Models

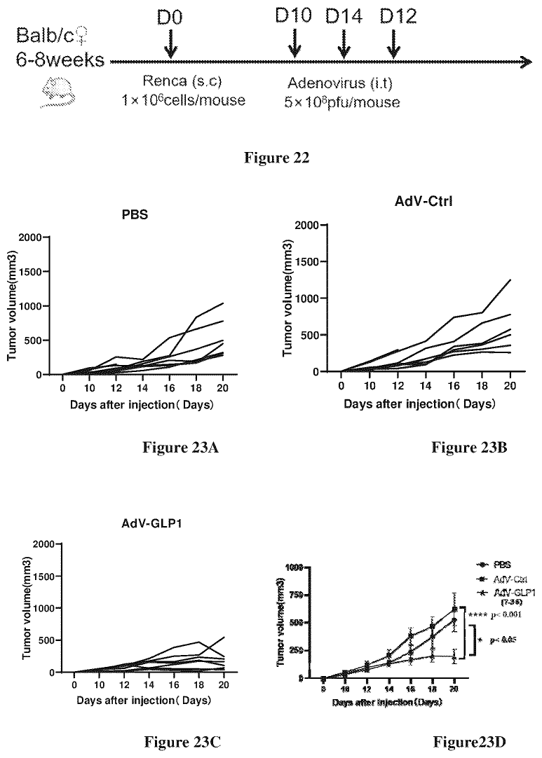

6-8 weeks old Balb/c mice were selected to establish a subcutaneous tumor model at the right flank with renca cells suspended in 10% matrigel in PBS (1×10E6 cells/mice, day 0, 24 mice total, FIG. 22), and randomly divided into 3 groups after the tumor size reached about 100 mm³ on day 10: A negative control group treated with PBS only (PBS, 0.1 mL), a control Ad5 group treated with Ad5 control (AdV-Ctrl, 5×10E8 pfu/dose), and a treatment group treated with Ad5 incorporated with the target DNA AdV-GLP-1(7-36)-CMV/EF1a (AdV-GLP1(7-36), 5×10E8 pfu/dose), all treatments were injected intratumorally one dose every other day for three doses (FIG. 22). The tumor volumes were measured after natural death of the mice.

AdV-GLP-1(7-36)-CMV/EF1a treatment showed statistically significantly slower tumor growth rate (AdV-GLP1(7-36), FIGS. 23C-23D) than the control Ad5 (p<0.001, AdV-Ctrl, FIGS. 23B and 23D) and the negative control group treated with PBS only (p<0.05, PBS, FIGS. 23A and 23D).

### Example IV: CD8+T Cell-Mediated Antitumor Effects of the Embodiment of the Recombinant Ad5 Adenovirus of the Recombinant Ad5 Adenovirus AdV-GLP-1(7-36)-CMV/EF1a

AdV-GLP-1(7-36)-CMV/EF1a was prepared according to the method of Example I with the target protein having a sequence of SEQ ID NO.: 1, a target DNA having a sequence of SEQ ID NO.: 5. The viral replication, expression of GLP-1 receptor agonist, and biological activity of the GLP-1 receptor agonist expressed were validated according to the method of Example II.

IV(A): AdV-GLP-1(7-36)-CMV/EF1a Treatments after Anti-CD8, Anti-CD4 or Anti-NK Treatment.

Pancreatic carcinoma cells were implanted subcutaneously into the right flanks of C57B6 mice. The mice were divided randomly into various treatment groups when their tumor volumes reached 100 mm³, and then received injections of: 1) PBS (PBS, 0.1 mL/dose) intratumorally; 2) control AdV (AdV-Ctrl, 5×10E8 ifu/dose) intratumorally; or 3) anti-CD8 intraperitoneally, anti-CD4 intraperitoneally, or anti-NK intraperitoneally, and then followed by intratumoral injection of AdV-GLP-1(7-36)-CMV/EF1a (5×10E8 ifu/dose), wherein the intratumoral injections were administered one dose every other day, two doses total. Then tumor volume and survival were monitored. N=6-8 mice for each group.

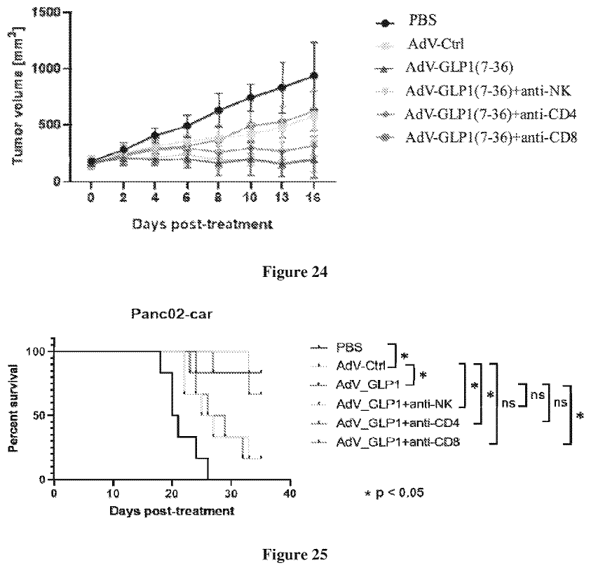

The group treated with AdV-GLP-1(7-36)-CMV/EF1a only (AdV-GLP1(7-36), FIG. 24) and the groups treated with anti-CD4 (GLP1(7-36)+anti-CD4, FIG. 24) or anti-NK (GLP1(7-36)+anti-NK, FIG. 24) before the AdV-GLP-1(7-36)-CMV/EF1a treatment showed slower tumor growth rate than the group treated with anti-CD8 (GLP1(7-36)+anti-CD8, FIG. 24) before the AdV-GLP-1(7-36)-CMV/EF1a treatment, the control Ad5 (AdV-Ctrl, FIG. 24) and the negative control group treated with PBS only (PBS, FIG. 24).

The group treated with anti-CD8 (GLP1(7-36)+anti-CD8, FIG. 25) before the AdV-GLP-1(7-36)-CMV/EF1a treatment did not show statistical significance in survival rate compared with the group treated with the control Ad5 (AdV-Ctrl, FIG. 25) but showed statistically significantly lower survival rate compared to the group treated with AdV-GLP-1(7-36)-CMV/EF1a only (AdV-GLP1(7-36), FIG. 25).

The group treated with AdV-GLP-1(7-36)-CMV/EF1a only (AdV-GLP1(7-36), FIG. 25) and the groups treated with anti-CD4 (GLP1(7-36)+anti-CD4, FIG. 25) or anti-NK (GLP1(7-36)+anti-NK, FIG. 25) before the AdV-GLP-1(7-36)-CMV/EF1a treatment showed statistically significantly longer survival rate compared to the group treated with the control Ad5 (*: p<0.5, AdV-Ctrl, FIG. 25). The groups treated with anti-CD4 (GLP1(7-36)+anti-CD4, FIG. 25) or anti-NK (GLP1(7-36)+anti-NK, FIG. 25) before the AdV-GLP-1(7-36)-CMV/EF1a treatment did not show statistically significant difference in survival rate compared to the group treated with AdV-GLP-1(7-36)-CMV/EF1a only (AdV-GLP1(7-36), FIG. 25).

### IV(B): AdV-GLP-1(7-36)-CMV/EF1a Treatments Significantly Enhanced Antitumor Activity and Reduces Exhaustion of CD8+T Cells.

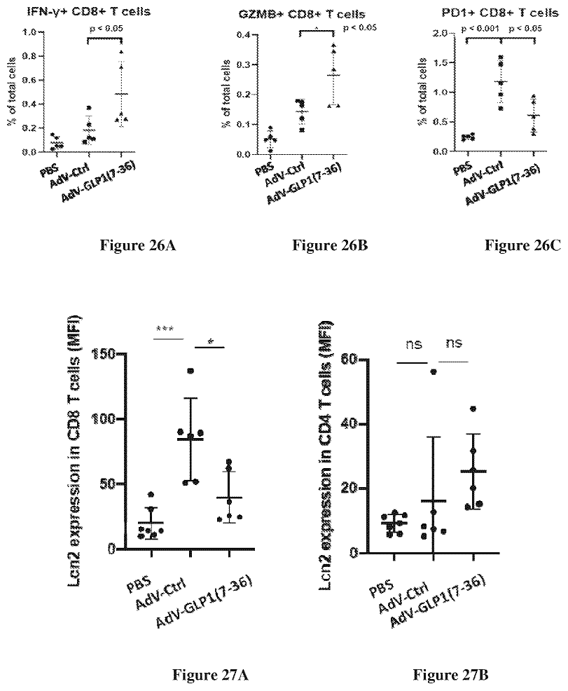

Pancreatic carcinoma cells were implanted subcutaneously into the right flanks of C57B6 mice. The mice were divided randomly into various treatment groups when their tumor volume reached about 200 mm³, then the mice received intratumoral injection of AdV-GLP-1(7-36)-CMV/EF1a (5×10E8 ifu/dose), control Ad5 (5×10E8 ifu/dose), or PBS (0.1 mL/dose) once dose every other day for two doses. Then 48 hrs after the second dose, their tumor tissues were isolated and singles cells were obtained for determination of specific marker as indicated in FIG. 26A (IFN-γ+CD8+ T cells), FIG. 26B (GZMB+CD8+ T cells) and FIG. 26C (PD1+CD8+ T cells). N=5 mice each group. Statistical significances were observed between the groups treated with AdV-GLP-1(7-36)-CMV/EF1a (AdV-GLP1(7-36), FIGS. 26A-26C) and control Ad5 (Adv-Ctrl, FIGS. 26A-26C).

Pancreatic carcinoma cells were implanted subcutaneously into the right flanks of C57B6 mice. The mice were divided randomly into various treatment groups when tumor volume reached to 100 mm³, and received injections of: 1) PBS (PBS, 0.1 mL/dose) intratumorally; 2) control AdV (AdV-Ctrl, 5×10E8 ifu/dose) intratumorally; or 3) anti-CD8 (200 μg) intraperitoneally, anti-CD4 (200 μg) intraperitoneally, or anti-NK (200 μg) intraperitoneally, and then followed by intratumoral injection of AdV-GLP-1(7-36)-CMV/EF1a (5×10E8 ifu/dose), wherein the intratumoral injections were administered one dose every other day, 2 doses total. Then tumor volume and survival were monitored. N=6-8 mice for each group.

T cells were isolated from the tumor tissues obtained supra (48 hours after the second dose of treatment of AdV-GLP-1(7-36)-CMV/EF1a, control Ad5, or PBS) and subjected to FACScan for characterizations.

Lipocalin 2 (LCN2), also known as Neutrophil Gelatinase-Associated Lipocalin (NGAL), is a secretory protein of the lipocalin superfamily. LCN2 is involved in many inflammatory diseases. LCN2 was shown to contribute to neutrophils recruitment and the CD8 T cell inhibition. AdV-GLP-1(7-36)-CMV/EF1a treatment enhanced antitumor responses of CD8+T cells by statistically significantly reducing LCN2 expression in CD8+ T cells (*: p<0.05, AdV-GLP1(7-36), FIG. 27A) compared to control Ad5 treatment (AdV-Ctrl, FIG. 27A), and control Ad5 treatment (AdV-Ctrl, FIG. 27A) increased Lcn2 expression in CD8+ T cells statistically significantly compared with the PBS group (***: p<0.0001, PBS, FIG. 27A); while AdV-GLP-1(7-36)-CMV/EF1a treatment showed no statistical significance in LCN2 expression in CD4+ T cells (AdV-GLP1(7-36), FIG. 27B) compared to control Ad5 treatment (AdV-Ctrl, FIG. 27B).

IV(C): GLP1 (7-36) or GLP1 Analogue (Liraglutide) Significantly Reduced LCN2 Expression in CD8+T Cells Cultured with Panc02 Cells

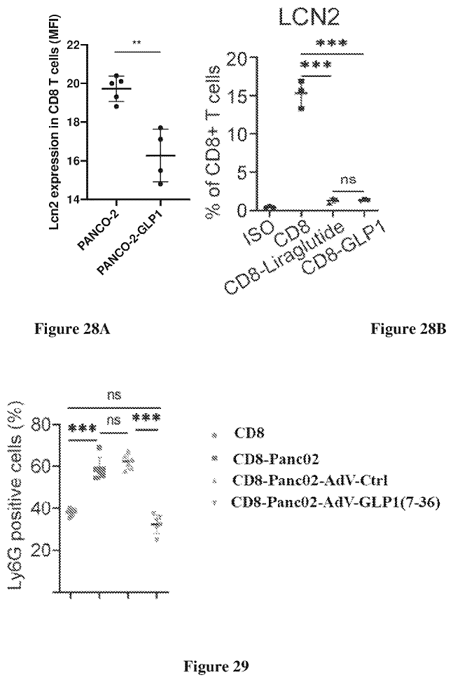

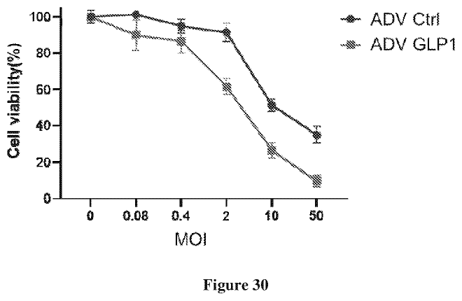

Panc02 cells were cocultured with CD8+ T cells with or without GLP1(7-36) for 24 h, then T cells were subjected to flow cytometry for determination of LCN2 expression (FIG. 28A). The presence of GLP-1(7-36) statistically significantly reduced Lcn2 expression in CD8+ T cells (**: p<0.01, PANCO-2-GLP1, FIG. 28A) compared to Panc02 cells cultured with CD8+ T cells only (PANCO-2, FIG. 28A).

Panc02 cells were cultivated in DMEM for 48 h, then supernatant was harvested and added into CD8+ T cells for further 24 h cultivation in the presence or absence of GLP1(7-36) or GLP1 analogue (Liraglutide) followed by FACScan for Lcn2 determination (FIG. VI(B)-3B). The presence of GLP-1(7-36) (***: p<0.001, CD8-GLP1, FIG. 28B) or liraglutide (***: p<0.001, CD8-GLP1, FIG. 28B) statistically significantly reduced Lcn2 expression in CD8+ T cells compared to Panc02 cells cultured with CD8+ T cells only (CD8, FIG. 28B), while no significant difference was observed between the reduction of Lcn2 expression in CD8+ T cells in the presence of GLP-1(7-36) and liraglutide (FIG. 28B).

### IV(D): AdV-GLP-1(7-36)-CMV/EF1a Significantly Inhibited CD8+ T Cell-Mediated Recruitment of Neutrophils In Vitro

Panc02 cells were infected with AdV-GLP-1(7-36)-CMV/EF1a, control Ad5, or PBS for 24 h followed by co-cultivation of CD8+ T cells isolated from tumor tissue for another 24 h. Then the CD8+T cells (in the cell suspension) were harvested and added into the lower-compartment of a transwell, and neutrophils isolated from the mice were added into the upper-compartment. 4 h later, the cell suspension from the lower-compartment were collected and subjected to determination of Ly6G neutrophil marker (FIG. 29).

AdV-GLP-1(7-36)-CMV/EF1a (CD8-Panc02-AdV-GLP1(7-36), FIG. 29) significantly inhibited CD8+ T cell-mediated recruitment of neutrophils in vitro compared to control Ad5 treatment (***: p<0.001, CD8-Panc02-AdV-Ctrl, FIG. 29).

GLP1 analogue (Liraglutide) treatment by intratumoral injection was shown before to induce nature killer (NK) cells-mediated antitumor effect in hepatocellular carcinoma. Unexpectedly, the recombinant adenovirus AdV-GLP-1(7-36)-CMV/EF1a which expresses GLP-1(7-36) (prepared according to Example I) induced CD8+T but not NK cell-mediated antitumor effect in pancreatic carcinoma. AdV-GLP-1(7-36)-CMV/EF1a also significantly reduced expression of LCN2 in CD8+T cells and significantly decreased intratumoral infiltration of neutrophils compared to the control Ad5 treatment. Note that intratumoral infiltration of neutrophils may exert immunosuppression in certain solid tumors.

### Example V. In Vitro and In Vivo Anti-Tumor Effects of the Embodiment of the Recombinant Ad5 Adenovirus AdV-GLP-1(7-37/A8G)-CMV

AdV-GLP-1(7-37/A8G)-CMV was prepared according to the method of Example I(A) with the target protein having a sequence of SEQ ID NO.: 2, a target DNA having a sequence of SEQ ID NO.: 6. The viral replication, expression of GLP-1 receptor agonist, and biological activity of the GLP-1 receptor agonist expressed were validated according to the method of Example II.

### V(A). In Vitro Anti-Tumor Effects of AdV-GLP-1(7-37/A8G)-CMV on Non-Small Lung Cancer Cells

A549 lung cancer cells were infected with AdV-GLP-1(7-37/A8G)-CMV and control Ad5 viruses with the MOI of 1-100, respectively. After 72 h, cell viability was measured using MTT to evaluate the tumoricidal effect of Ad5 GLP1.

### V(B). In Vivo Anti-Tumor Effects of AdV-GLP-1(7-37/A8G)-CMV on Pancreatic Carcinoma Animal Models

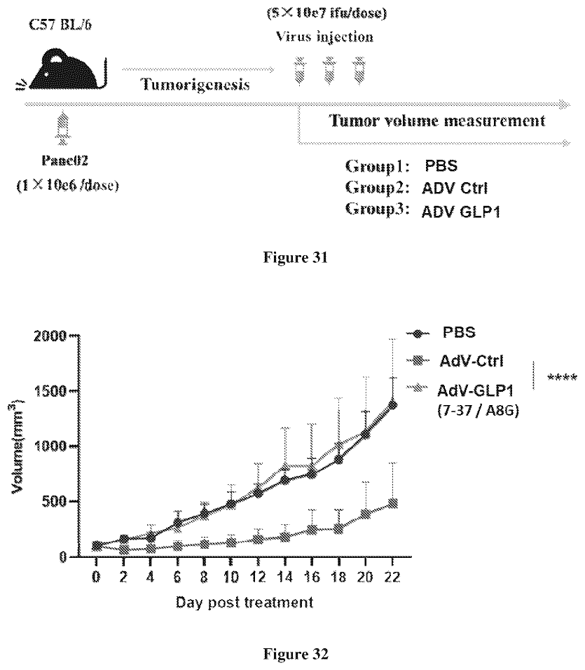

6-8 weeks old C57BL/6 mice were selected to establish a subcutaneous tumor model at the right flank with Panc02 cells suspended in 10% matrigel in PBS (1×10E6 cells/mice, 18 mice total, FIG. 31), and randomly divided into 3 groups after the tumor size reached about 100 mm³: a negative control group treated with PBS only (PBS, 0.1 mL), a control Ad5 group treated with Ad5 control (AdV-Ctrl, 5×10E7 ifu/dose), and a treatment group treated with Ad5 incorporated with the target DNA AdV-GLP-1(7-37/A8G)-CMV (AdV-GLP1(7-37/A8G), 5×10E7 ifu/dose), all treatments were injected intratumorally every other day for three doses (FIG. 31). The tumor volume and body weight were measured and the survival period was recorded after natural death of the mice.

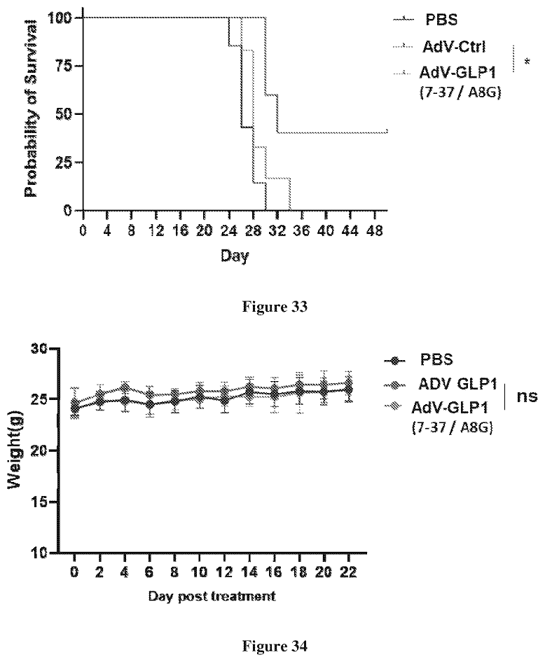

AdV-GLP-1(7-37/A8G)-CMV treatment showed statistically significantly slower tumor growth rate (AdV-GLP1(7-37/A8G), FIG. 32) than the control Ad5 (p<0.001, AdV-Ctrl, FIG. 32); AdV-GLP-1(7-37/A8G)-CMV treatment showed slower tumor growth rate than the negative control group treated with PBS only (PBS, FIG. 32). AdV-GLP-1(7-37/A8G)-CMV treatment showed statistically significantly improved survival rate (AdV-GLP1(7-37/A8G), FIG. 33) than the control Ad5 (p<0.05, AdV-Ctrl, FIG. 33); AdV-GLP-1(7-37/A8G)-CMV treatment showed improved survival rate than the negative control group treated with PBS only (PBS, FIG. 33). No significant change of body weights was observed among the three treatment groups (FIG. 34).

### Example VI. In Vivo Anti-Tumor Effects of the Embodiment of the Recombinant Ad5 Adenovirus AdV-GLP-1(7-36)-4GS-PLGF2-CMV/EF1a and AdV-PLGF2-4GS-GLP-1(7-36)-CMV/EF1a

AdV-GLP-1(7-36)-4GS-PLGF2-CMV/EF1a and AdV-PLGF2-4GS-GLP-1(7-36)-CMV/EF1a were prepared according to the method of Example I(B) with the target protein having a sequence of SEQ ID NO.: 3 and the target DNA having a sequence of SEQ ID NO.: 7 for AdV-GLP-1(7-36)-4GS-PLGF2-CMV/EF1a, and the target protein having a sequence of SEQ ID NO.: 4 and the target DNA having a sequence of SEQ ID NO.: 8 for AdV-PLGF2-4GS-GLP-1(7-36)-CMV/EF1a. The viral replication, expression of GLP-1 receptor agonist, and biological activity of the GLP-1 receptor agonist expressed were validated according to the method of Example II.

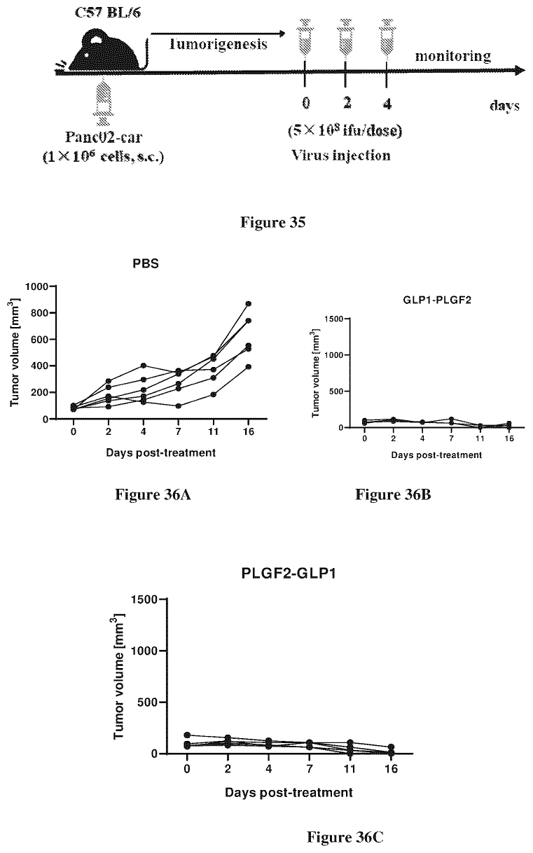

6-8 weeks old C57BL/6 mice were selected to establish a subcutaneous tumor model at the right flank with panc02-car cells suspended in 10% matrigel in PBS (1×10E6 cells/mice, 15 mice total, FIG. 35), and randomly divided into 3 groups after the tumor size reached about 100 mm³: a negative control group treated with PBS only (PBS, FIG. 36A), a control Ad5 group treated with Ad5 control (AdV-Ctrl), and a treatment group treated with Ad5 incorporated with the target DNA, AdV-GLP-1(7-36)-4GS-PLGF2-CMV/EF1a (AdV-GLP1(7-36)-PLGF2, FIG. 36B) or AdV-PLGF2-4GS-GLP-1(7-36)-CMV/EF1a (AdV-PLGF2-GLP1(7-36), FIG. 36C). The mice were injected intratumorally with the 5×10E8 ifu/dose of virus or 0.1 mL PBS every other day for three doses (FIG. 35). The tumor volumes were measured.

Both AdV-GLP-1(7-36)-4GS-PLGF2-CMV/EF1a (FIG. 36B) and AdV-PLGF2-4GS-GLP-1(7-36)-CMV/EF1a (FIG. 36C) treatment showed slower tumor growth rate than the negative control group treated with PBS only (FIG. 36A).

1. A recombinant adenovirus or adenoviral vector comprising a first promoter, an E1A early activation replication element, an optional second promoter, and a target nucleotide sequence encoding a target protein comprising a glucagon-like peptide-1 (GLP-1) receptor agonist,
the first promoter being a constitutive promoter; and:
the optional second promoter being a constitutive promoter which may be the same as or different from the first promote.

2. The recombinant adenovirus or adenoviral vector of claim 1, the constitutive promoter being selected from the group consisting of CMV promoter, and EF1a promoter.

3. The recombinant Ad5 adenovirus or adenoviral vector of claim 1, the first promoter being CMV promoter.

4. The recombinant adenovirus or adenoviral vector of claim 1, the second promoter being EF1a promoter.

5. The recombinant adenovirus or adenoviral vector of claim 1, further comprising a nucleotide sequence encoding a IL-2 signal peptide.

6. The recombinant adenovirus or adenoviral vector of claim 1, the GLP-1 receptor agonist comprising an amino acid sequence with 80% homology or higher to a sequence selected from the group consisting of bioactive fragment of GLP-1 (e.g., GLP1(7-36), GLP1(7-37)) and GLP-1 analogs.

7. The recombinant adenovirus or adenoviral vector of claim 1, the target protein comprises the GLP-1 receptor agonist and a tumor homing protein.

8. The recombinant adenovirus or adenoviral vector of claim 1, the GLP-1 receptor agonist and the tumor homing protein is linked by a linker of (GGGGS)ₙ, n=1, 2, 3, 4, 5, 6, 7, 8, 9, or 10.

9. The recombinant adenovirus or adenoviral vector of claim 1, the GLP-1 analog is a GLP-1 fragment with one or more substituted amino acids, one or more omitted amino acids, and/or one or more additional amino acids.

10. The recombinant adenovirus or adenoviral vector of claim 1, the GLP-1 receptor agonist being a secretory glucagon-like peptide-1 protein.

11. The recombinant adenovirus or adenoviral vector of claim 1, the recombinant adenovirus or adenoviral vector causing one or more effects on one or more tumors the recombinant adenovirus or adenoviral vector contacts, the one or more effects being selected from: tumor growth inhibition, tumor recession, and prolonged survival.

12. The recombinant adenovirus or adenoviral vector of claim 1, the adenovirus or adenoviral vector inducing CD8+T-mediated antitumor effect in a tumor of a subject when administered to the subject.

13. A pharmaceutical composition comprising the recombinant adenovirus or adenoviral vector of claim 1, and a pharmaceutically acceptable carrier.

14. A method of inducing CD8+T cell-mediated antitumor effects in a subject comprising administering to the subject a therapeutically effective amount of the recombinant adenovirus or adenoviral vector of claim 1.

15. A method for treating or preventing one or more cancers in a subject comprising administering to the subject a therapeutically effective amount of the recombinant adenovirus or adenoviral vector of claim 1.

16. The method according to claim 15, the one or more cancers are selected from the group consisting of liver cancer, pancreatic cancer, colon cancer, glioma, lung cancer, esophageal carcinoma, gastric cancer, breast cancer, ovarian cancer, prostate cancer, kidney cancer, squamous cell carcinoma of head and neck, melanoma, multiple myeloma, and lymphoma.

17. The method according to claim 16, the lung cancer is non-small lung cancer.

18. The method of claim 14, further comprising inhibiting tumor invasion and/or metastasis.

19. The method of claim 14, further comprising one or more effects selected from the group consisting of: reducing expression of LCN2 in CD8+T cells, reducing intratumoral infiltration of neutrophils, inhibiting tumor inflammation pathways, inhibiting IDO-1, and/or restoring anti-tumor immune surveillance.

20. The method of claim 14, further comprising delaying a cachexia progress of the subject.

21. The method of claim 14, further comprising prolonging life span of the subject.

22. The method of claim 14, the recombinant adenovirus or adenoviral vector or the pharmaceutical composition being administered in a single-drug therapy, adjuvant therapy, or combination therapy.

23. The method according to claim 14, the recombinant adenovirus or adenoviral vector or the pharmaceutical composition being administered intratumorally.
